(19) United States
(12) Patent Application Publication
(10) Pub. No.: US 2023/0329592 A1
(43) Pub. Date: Oct. 19, 2023

(54) ABRASION PROTECTED MICRONEEDLE AND INDWELLING EAB SENSORS

(71) Applicant: University Of Cincinnati, Cincinnati, OH (US)

(72) Inventor: Jason Charles Heikenfeld, Cincinnati, OH (US)

(21) Appl. No.: 18/027,391

(22) PCT Filed: Sep. 24, 2021
(86) PCT No.: PCT/US2021/051865

§ 371 Date: Mar. 21, 2023

Related U.S. Application Data
(60) Provisional application No. 63/150,634, filed on Feb. 18, 2021.
(60) Provisional application No. 63/082,810, filed on Sep. 24, 2020.

(51) Int. Cl. A61B 5/145; A61B 5/1473; A61B 5/00

(52) U.S. Cl. CPC A61B 5/14514; A61B 5/1473; A61B 5/685; A61B 2562/125

(57) ABSTRACT

A continuous sensing device for measuring at least one analyte in interstitial fluid is provided. The device (100) includes at least one feature (114) configured to be inserted into a body, and specifically, the at least one feature configured to be inserted into a skin (12) of the body. The at least one feature is at least partially coated with at least one electrode (120) functionalized with an aptamer sensing monolayer layer (122), and the aptamer sensing monolayer layer includes an aptamer with attached redox couples and passivating material. The at least one feature is configured to provide at least one of a resistance to abrasion effect or a pressure effect for the aptamer sensing monolayer when the feature is placed into the body.

### CROSS-REFERENCE TO RELATED APPLICATIONS

This application claims priority to, and the benefit of the filing date of, U.S. Provisional Application No. 63/082,810 filed Sep. 24, 2020 and U.S. Provisional Application No. 63/150,634 filed Feb. 18, 2021, the disclosures of which are incorporated by reference herein in their entirety.

### TECHNICAL FIELD

The present invention relates to the use of electrochemical, aptamer-based (E-AB) sensors.

### BACKGROUND OF THE INVENTION

This section is intended to introduce the reader to various aspects of art that may be related to various aspects of the present invention, which are described and/or claimed below. This discussion is believed to be helpful in providing the reader with background information to facilitate a better understanding of various aspects of the present invention. Accordingly, it should be understood that these statements are to be read in this light, and not as admissions of prior art.

Interstitial fluid contains many of the same analytes as blood and often at comparable concentrations. As a result, interstitial fluid presents an alternative biofluid to blood for detection of analytes such as glucose for diabetes monitoring. Commonly employed practices for continuous monitoring of glucose in interstitial fluid include (1) in-dwelling sensors, where a needle is utilized to insert the sensor into the dermis of the skin, and (2) ex-vivo sensors, where micro-needles penetrate the surface of the skin and the analyte is coupled from interstitial fluid to the sensor by diffusion to the sensor. In products, and in research, the biosensing of analytes in interstitial fluid monitoring has been dominated by metabolite analytes because electrochemical enzymatic sensors are readily available and well developed for these compounds, and because metabolites are found at generally high concentrations (mM) which simplifies their detection. Even so, use of an enzymatic sensor with a microneedle device has not yet seen commercial success, at least in part because microneedles have difficulty in keeping continuous contact with the dermis. For an enzymatic sensor, which relies on a diffusive flux of analyte to the sensor, any change in coupling between the microneedles and the dermis of the skin would result in a false-change in measured glucose signal. Even in-dwelling sensors can suffer from motion artifacts as an enzymatic sensor’s position in the dermis can impact diffusive flux of glucose to the sensor.

Affinity-based sensors, such as electrochemical or optical aptamers, are known to be inherently reversible (and thus truly continuous) and known to provide ranges of detections in the µM or lower ranges in biofluids such as whole blood. These sensors, however, are quite different than enzymatic sensors, which metabolize and therefore consume the analyte. This is because affinity sensors require equilibration of analyte concentration with the sensor itself, and have a known binding affinity for the target analyte. However, such an approach is ill-suited to a continuous wearable format, as unlike enzymatic sensors, electrochemical aptamer sensors are extremely sensitive to abrasion or pressure against the sensor surface.

### SUMMARY OF THE INVENTION

Certain exemplary aspects of the invention are set forth below. It should be understood that these aspects are presented merely to provide the reader with a brief summary of certain forms the invention might take and that these aspects are not intended to limit the scope of the invention. Indeed, the invention may encompass a variety of aspects that may not be explicitly set forth below.

Many of the drawbacks and limitations stated above can be resolved by creating novel and advanced interplays of chemicals, materials, sensors, electronics, microfluidics, algorithms, computing, software, systems, and other features or designs, in a manner that affordably, effectively, conveniently, intelligently, or reliably brings sensing technology into proximity with sample fluids containing at least one analyte of interest to be measured.

Aspects of the disclosed invention are directed to electrochemical aptamer-based sensors that have resistance to the confounding effects of abrasion and pressure. One particular aspect of the present invention is directed to a continuous sensing device for measuring at least one analyte in interstitial fluid. The device includes at least one feature configured to be inserted into a body, and specifically, the at least one feature may be configured to be inserted into a skin of the body. The at least one feature is at least partially coated with at least one electrode functionalized with an aptamer sensing monolayer layer, and the aptamer sensing monolayer layer includes an aptamer with attached redox couples and passivating material. The at least one feature is configured to provide at least one of a resistance to abrasion effect or a pressure effect for the aptamer sensing monolayer when the feature is placed into the body.

In certain embodiments, the feature is a porous or grooved surface of a microneedle. In addition, in certain embodiments including a microneedle, only the inside of the electrode is coated.

In another embodiment, the feature includes a membrane covering the electrode. In another embodiment, the feature includes the aptamer sensing monolayer that is added onto the electrode.

Another aspect of the present invention is directed to a method of fabricating a continuous sensing device for measuring at least one analyte in interstitial fluid. The device contains at least one feature that is coated at least in part with at least one electrode that is functionalized with aptamers and attached redox couples to electrochemically measure the analyte. The method involves fabricating the at least one feature that provides at least one of abrasion resistance or pressure resistance when placed into the dermis of the skin. In one embodiment, the method also involves coating an electrode, an aptamer sensing layer, or both on the feature, and then removing the electrode and/or the aptamer sensing layer from all regions of the feature where variable pressure or abrasion could be problematic. In another embodiment, an aptamer sensing layer, blocking layers, or both are applied after partial removal of the microneedle feature. In one embodiment, an aptamer sensing layer, blocking layers, or both are applied before partial removal of the microneedle feature. In another embodiment, an electrode is coated with a porous protected material, after which an aptamer sensing layer, blocking layers, or both are applied to the remaining electrode surface.

### BRIEF DESCRIPTION OF THE DRAWINGS

The objects and advantages of the disclosed invention will be further appreciated in light of the following detailed descriptions and drawings in which:

FIG. 1A is a cross-sectional view of a device according to an embodiment of the disclosed invention.

FIG. 1B is a cross-sectional view of a portion of a microneedle according to an embodiment of the disclosed invention.

FIG. 1C is a cross-sectional view of a portion of a microneedle according to an embodiment of the disclosed invention.

FIG. 2A is a is a cross-sectional view of a portion of a microneedle according to an embodiment of the disclosed invention.

FIG. 2B is a cross-sectional view of a portion of a microneedle according to an embodiment of the disclosed invention.

FIG. 3 is a cross-sectional view of a portion of a microneedle according to an embodiment of the disclosed invention.

### DEFINITIONS

As used herein, the term “about,” when referring to a value or to an amount of mass, weight, time, volume, pH, size, concentration or percentage is meant to encompass variations of ±20% in some embodiments, ±10% in some embodiments, ±5% in some embodiments, ±1% in some embodiments, ±0.5% in some embodiments, and ±0.1% in some embodiments from the specified amount, as such variations are appropriate to perform the disclosed method.

As used herein, the term “aptamer” means a molecule that undergoes a conformation change as an analyte binds to the molecule, and which satisfies the general operating principles of the sensing method as described herein. Such molecules are, e.g., natural or modified DNA, RNA, or XNA oligonucleotide sequences, spiegelmers, peptide aptamers, and affimers. Modifications may include substituting unnatural nucleic acid bases for natural bases within the aptamer sequence, replacing natural sequences with unnatural sequences, or other suitable modifications that improve sensor function.

As used herein, the term “sensing monolayer” means aptamers that are functionalized with a redox tag, such as methylene blue or other redox tag, and attached onto an electrode such as gold by thiol linkage or other suitable chemistry, and the space in between the aptamers on the electrode passivated by a passivating material such as mercaptohexanol or other suitable passivating material.

The devices and methods described herein encompass the use of sensors. A “sensor,” as used herein, is a device that is capable of measuring the concentration of a target analyte in solution. As used herein, an “analyte” may be any inorganic or organic molecule, for example: a small molecule drug, a metabolite, a hormone, a peptide, a protein, a carbohydrate, a nucleic acid, or any other composition of matter. The target analyte may comprise a drug. The drug may be of any type, for example, including drugs for the treatment of cardiac system, the treatment of the central nervous system, that modulate the immune system, that modulate the endocrine system, an antibiotic agent, a chemotherapeutic drug, or an illicit drug. The target analyte may comprise a naturally-occurring factor, for example a hormone, metabolite, growth factor, neurotransmitter, etc. The target analyte may comprise any other species of interest, for example, species such as pathogens (including pathogen induced or derived factors), nutrients, and pollutants, etc.

### DETAILED DESCRIPTION OF THE INVENTION

One or more specific embodiments of the present invention will be described below. In an effort to provide a concise description of these embodiments, all features of an actual implementation may not be described in the specification. It should be appreciated that in the development of any such actual implementation, as in any engineering or design project, numerous implementation-specific decisions must be made to achieve the developers’ specific goals, such as compliance with system-related and business-related constraints, which may vary from one implementation to another. Moreover, it should be appreciated that such a development effort might be complex and time consuming, but would nevertheless be a routine undertaking of design, fabrication, and manufacture for those of ordinary skill having the benefit of this disclosure.

Certain embodiments of the disclosed invention show sensors as simple individual elements. It is understood that many sensors require two or more electrodes, reference electrodes, or additional supporting technology or features which are not captured in the description herein. Sensors measure a characteristic of an analyte. Sensors are preferably electrical in nature, but may also include optical, chemical, mechanical, or other known biosensing mechanisms. Sensors can be in duplicate, triplicate, or more, to provide improved data and readings. Sensors may provide continuous or discrete data and/or readings. Certain embodiments of the disclosed invention show sub-components of what would be sensing devices with more sub-components needed for use of the device in various applications, which are known (e.g., a battery, antenna, adhesive), and for purposes of brevity and focus on inventive aspects, such components may not be explicitly shown in the diagrams or described in the embodiments of the disclosed invention. All ranges of parameters disclosed herein include the endpoints of the ranges.

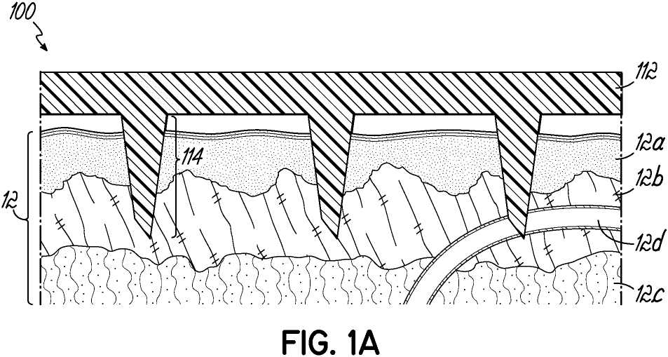

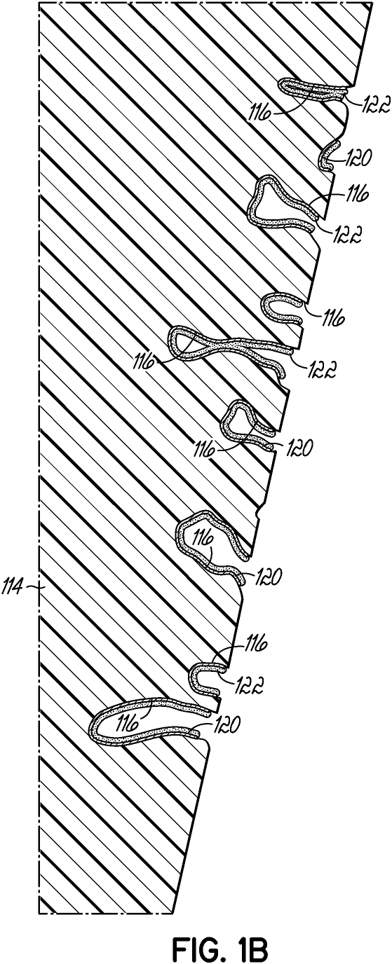

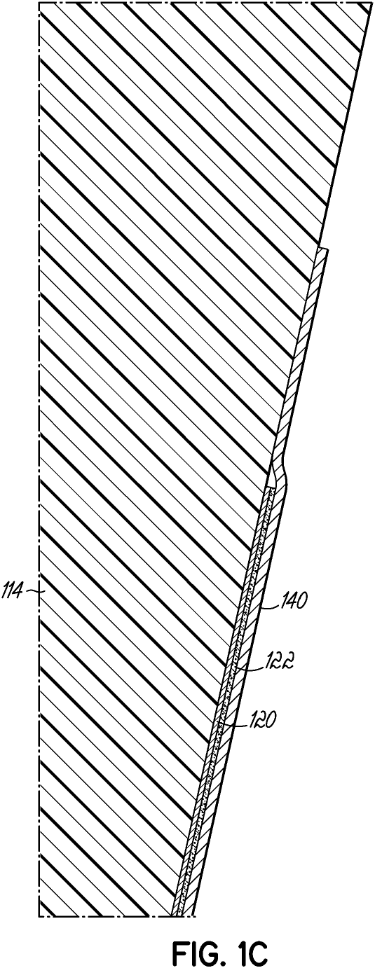

With reference to FIG. 1A, in an embodiment of the disclosed invention, a device 100 is placed partially in-vivo into the skin 12 comprised of the epidermis 12a, dermis 12b, and the subcutaneous or hypodermis 12c. A portion of the device 100 accesses or is configured to access fluids such as interstitial fluid from the dermis 12b and/or blood from a capillary 12d. Access is provided, for example, by element 112, which includes features 114. The features 114 may include microneedles formed of metal, polymer, semiconductor, glass, or other suitable material. Alternately, feature 114 could be a single indwelling needle that can be several mm in length or more, a flexible circuit formed on Kapton film, or any material or component which can reliably be or is configured to be inserted into the dermis or deeper (hypodermis, or deeper yet) for purpose of sensing analytes in the body.

As has been noted above, aspects of the present invention include the use of aptamers (such as in electrochemical aptamer-based sensors). However, if a feature 114 were to be coated with an aptamer on its surface, there are several challenges presented by insertion into the body such as the dermis 12b. Firstly, tissue can abrade the surface and remove the monolayer of aptamer and blocking layer such as mercaptohexanol which are both typically thiol bonded to a gold surface. Secondly, and in some instances even more potentially challenging, pressure of tissue such as collagen against the sensor can cause signal changes from the aptamer, for example by physical pressure or by the negatively charged membrane proteins on surface of many types of tissue that can cause electronic or steric repulsion and could change the distance of the redox tag on the aptamer (e.g., methylene blue) with the electrode that transfers charge to/from the redox tag. Cellular or other materials in the dermis can also interfere with aptamers. Drawbacks such as these were discussed above in the “Background” section.

Aspects of the present invention eliminate these drawbacks. With reference now to FIG. 1B, at least a portion of the feature 114 inserted into the dermis 12b is coated with an electrochemical electrode 120 such as gold, platinum, carbon, or other electrode material that is further functionalized with aptamer sensing monolayer 122. This example embodiment, shown in FIG. 1B, is abrasion and pressure resistant because the aptamer sensing monolayer 120 is not at the surface of the feature 114, but is protected within multiple pores or cavities or grooves 116 in the feature 114. Such a device can be fabricated as follows, as an example. In an embodiment, only the pores or grooves of the surface that are not exposed to tissue or cellular content are functionalized with the aptamer sensing monolayer. As shown in FIG. 1B, the electrode 120 and the aptamer sensing monolayer 122 are coupled to the feature 114 only within the grooves 116 located on the surface of the feature 114. In another embodiment, the aptamer sensing monolayer 122 is not exposed or not substantially exposed to tissue or cellular content in the body, and the electrode 120 coats both exposed and non-exposed portions of the least one feature 114 inserted into the skin. Furthermore, in this embodiment, the exposed portions of the electrode 120 are coated with the aptamer sensing monolayer. Feature 114 is made of a porous or grooved material such as ceramic or porous metal such as stainless steel that is coated with an electrode 120 coating by means such as electroplating or physical vapor deposition of a metal such as gold, platinum, or other electrically conductive materials such as carbon, diamond, conducting polymers, etc. Feature 114 and electrode 120 can then be sandblasted, surface polished with abrasive paper or a rubber or plastic sheet, or other techniques, to remove all highly exposed electrode 120 surfaces. In certain embodiments, the surface of feature 114 has grooves or channels 116 such that the electrode coating is continuous and connected. This brings a potential advantage that if feature 114 is electrically insulating then abrasion of its exposed surface in the dermis and exposure of its surface will not cause any background electrochemcial current which could cause sensor signal interference or decrease sensor signal to background current ratio. After electrode 120 is removed from exposed surface, the electrode 120 surface can then be functionalized with aptamer sensing monolayer 122. Aptamer sensing monolayer 122 may include one or more sensors and/or a sensing monolayer of aptamer and passivating layer materials such as mercaptohexanol as is conventionally performed for aptamer sensors. The feature 114 may be configured to provide at least one of a resistance to abrasion effect or a pressure effect. In an example, the feature 114 includes at least one material added onto the feature 114 wherein the material provides the configuration that provides at least one of a resistance to abrasion effect or a pressure effect for the aptamer sensing monolayer 122 when the feature 114 is placed into the body. For example, in an example, an electrode and/or sensing layer is coated, then removed in all regions where variable pressure or abrasion could be problematic. In an example, the aptamer and blocking layers 122 can be applied before or after partial removal of the electrode 120 material. Alternatively, or in addition, a biocompatible dissolvable material may be included with the feature 114, and may be the at least one material added onto the feature 114 to provide the configuration that provides at least one of a resistance to abrasion effect or a pressure effect for the aptamer sensing monolayer when the feature is placed into the body. As a result, aptamer sensing layers 122 would be protected from variable pressure or abrasion on or inside the body such as the stratum corneum of skin 12, or from cellular material in the epidermis 12a, or collagen in the dermis 12b, fat in the subcutaneous 12c, or other interfering materials in the body. As shown in FIG. 1B, the feature 114 may include a plurality of grooves 116 or pores, and the plurality of grooves 116 or pores may be configured to provide at least one of resistance to abrasion effects or pressure effects for the sensing monolayer 122 when placed into the body. Furthermore, the plurality of grooves 116 or pores may be coated with a membrane 140. Although in FIG. 1B the surface of feature 114 is shown to provide protection of the aptamer by virtue of grooves 116 or porosity of feature 114, any aspect of feature 114 that provides suitable provides abrasion or pressure protection may be included as an alternate embodiment of the present invention such as a rectangular microchannel, one or more cylindrical pores, or other suitable features that achieve resistance of the effects of abrasion and pressure on the sensing monolayer in the body.

Alternately, as shown in FIG. 1C, an electrode 120, such as gold, is coated on a surface of feature 114, an aptamer sensing monolayer 122, and a protective membrane 140 applied to protect the sensor from abrasion or pressure effects. The membrane 140 need not protect the sensor from fouling, but rather the membrane’s 140 main function is to simply protect the sensor from abrasion or physical contact with large materials in the body (cellular, tissue, etc.) that would interfere with the sensor signal. This membrane 140 protection approach, however, can be challenging, because the membrane 140 can translate motion or abrasion to the aptamer sensing monolayer 122 if the membrane 140 is loose on the surface in any manner or if the membrane 140 is compressible, which is true for many membranes especially those formed from hydrogels. Therefore, a membrane 140 may be applied as well (not shown) to a device similar to that of FIG. 1B such that pressure and abrasion resistance can be achieved, along with the option to have protective benefit of a membrane 140 against fouling species, proteases, and other sensor-damaging/confounding solutes in the body.

With further reference to the present invention, an alternate method of fabrication (not shown) is additive instead of subtractive like the previous taught method. In an embodiment, a surface is coated with a gold or other electrode, and a porous or non-continuous material is deposited onto the gold electrode, such as electrodeposited or spray-coated polymer such as electrodeposited photoresist or spray coated acrylic in a solvent such as acetone. This polymer then forms a porous yet protected network on the electrode, and the electrode can then be functionalized with aptamer and blocking layer on the remaining exposed electrode areas. In this method, an electrode is coated with a porous protecting material, after which the aptamer and blocking layers can be applied to the remaining electrode surface.

With further reference to the present invention, any of the embodiments as taught herein can be coated with a biocompatible dissolvable material such as sucrose, for example, the aptamer sensing monolayer 122 may be coated with a bio-compatible dissolvable material, denatured serum that is 5 kDa filtered, or other suitable material, to protect any surfaces until the device is inserted into the skin. The biocompatible dissolvable material can then be naturally removed (dissolved) by the body to reveal the sensor surface.

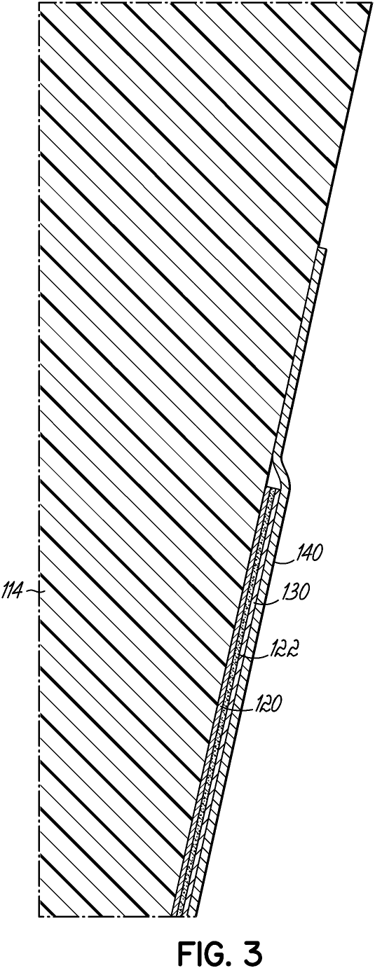

With reference to FIG. 3, as explained above, a membrane 140 may be applied as well to a device similar to that of FIG. 1B such that pressure and abrasion resistance can be achieved, along with the protective benefit of a membrane 140 against fouling species, proteases, and other sensor-damaging/confounding solutes in the body. Alternately, the membrane 140, such as a microfiltration membrane made of polyestersulphone (PES) or cellulose, and the aptamer sensing layer 120, could be physically separated by a spacer material 130 such as microbeads, photo-resist pillars such as SU-8 that can be fabricated onto the electrode 120, a dilute hydrogel such as polyacrylamide or agar, or other spacer material 130 to prevent the membrane 140 from abrading the aptamer sensing monolayer 122 or placing variable pressure against the aptamer sensing monolayer 122 that would cause false aptamer signal changes. The placement of this spacer material 130, as shown in FIG. 3, may be between membrane 140 and the aptamer sensing monolayer 122 as shown in FIG. 1C. For example, a sensing monolayer could be coated, then spacer balls in dissolvable material such as sucrose or trehalose coated, then the membrane coated on that, and the sucrose dissolved away during operation.

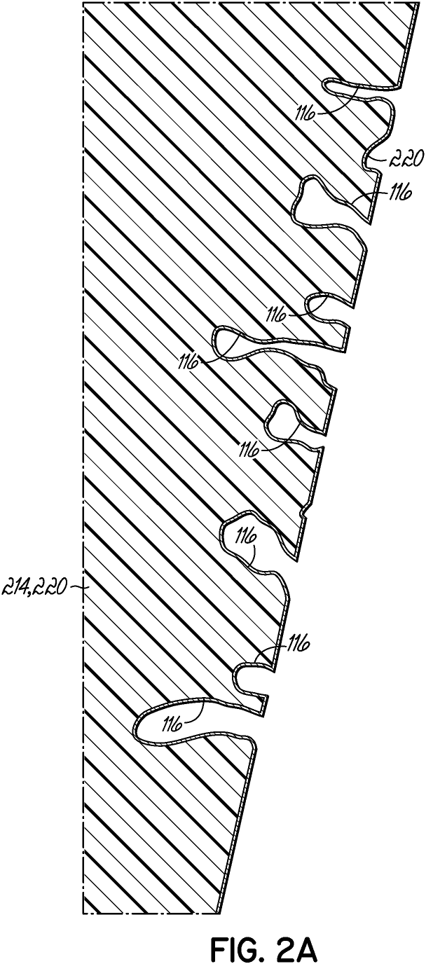

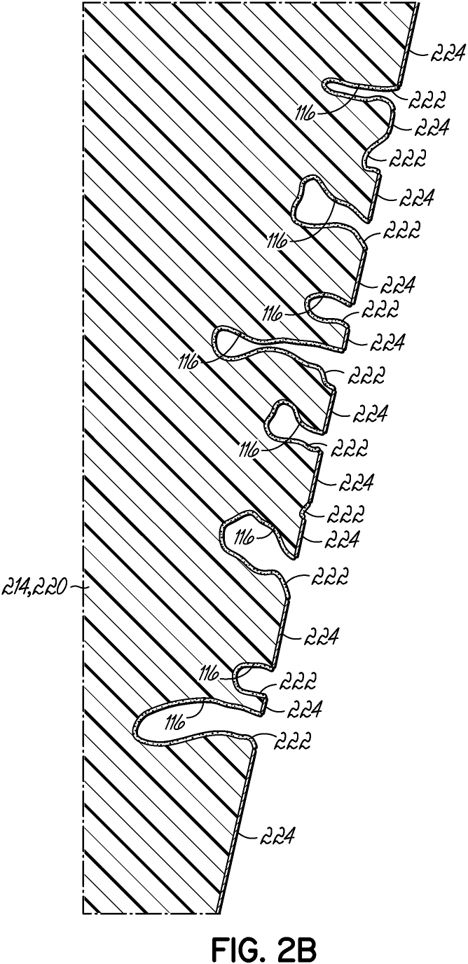

With further reference to the present invention, as illustrated in FIG. 2A where like numerals refer to like features, an alternate method of fabrication can be taught, where some or the entire surface of feature 214 is coated with an electrode 220 such as gold or feature 214 itself is gold. This electrode 220 surface is then functionalized with an aptamer sensing monolayer 222. Next, to avoid losing aptamer or mercaptohexanol when the device is in the body or to mitigate pressure effects on the aptamer in the body, the feature 214 coated with the electrode 220 is polished with a cloth, rubber, or other material to remove the aptamer sensing monolayer 222 from all exposed surfaces. Next the feature 214 is inserted into the body, and any exposed and non-functionalized/passivated surfaces of the feature 214 are then naturally passivated by endogeneous solutes in the body such as peptides, proteins, and other solutes in interstitial fluid and blood. In an embodiment, the exposed portions of the at least one feature 114 inserted into the skin are configured to absorb at least one endogenous solute found in the body. As a result, abrasion in the body will not cause loss of aptamer or passivation molecules into the body, which is also beneficial as it minimizes any signal change due to loss of aptamer or monolayer. Natural passivation of the surface of feature 214 coated with electrode 220 can be illustrated as material 224 in FIG. 2B. Alternately, the exposed feature 214 can be passivated before the feature 214, is placed into the body, for example by being passivated by material 224, which includes, in some embodiments, mercaptohexanol, thiol-linked polyethylene glycol, hexanethiol, or other suitable exogenous passivating material not found in the body.

With further reference to the present invention, any of the embodiments as taught herein can have various porosities for the electrode 120, 220. For example, porous gold can be formed by a simple procedure which involves an acidic treatment of a commercially available complex white-gold or gold-silver alloy. 24 hour HNO3 treatment can provide up to 12,400 times surface enlargement and resulted in a surface area of 14.2 m2/g. With use with aptamers, pores can typically be as small as allows freedom of movement for the aptamer (approximately 10 nm or larger). In an embodiment, the feature 114 includes an exposed area configured to be exposed to tissue or cellular content in the body after the feature is inserted into the body, and an unexposed area that is configured to be unexposed to tissue or cellular content in the body after the feature 114 is inserted into the body, and which carriers the aptamer sensing monolayer 122, 222. The ratio of protected and unexposed porous surface with the aptamer sensing monolayer 122, 222 to the surface of the electrode 120, 220 that is exposed and unprotected to abrasion can therefore be at least one of >1.3X, >3X, >10X, >30X, >100X. Alternately, porous electrodes can be electrodeposited, including for example for gold by adding a sufficient amount of ammonium chloride is added to the electrolyte as a hydrogen source during electrodeposition. Porous electrodes can also have such small porosity that they also, in effect, act as a protecting membrane by excluding larger solutes such as cells, albumin, enzymes, nucleases, etc. The ratio of protected and unexposed porous surface also applies to other embodiments, such as FIG. 1B, where the ratio of the aptamer sensing monolayer 122 to the surface of the feature 114 that is exposed and unprotected is at least one of >1.3X, >3X, >10X, >30X, >100X.

With further reference to the present invention, too great of a porosity could disable proper functionalization of the electrode. The first step in electrochemical aptamer electrode functionalization is to bond the redox-tagged aptamer to the electrode, often using chemistry such as thiol-linkage if the electrode is gold. Typically, aptamer densities range from ~10¹⁰ to 10¹²/cm². An ultra-porous surface would suffer from low aptamer densities in its deepest crevices and too high aptamer densities near the surface, because during wet functionalization of the electrode with aptamer the aptamers will link to nearby gold surfaces as they slowly diffuse into the porous electrode. This will often appear as a noisier signal or a sensor that does not properly respond to changes and analyte signal. Also, too high of electrode porosity can cause a long lag time for the sensor response as greater amount of analyte must diffuse to the aptamers and also diffuse through a more tortuous network of pores. Therefore, the present invention may utilize a ratio of protected porous surface to the surface of the electrode 120, 220 that is exposed and unprotected to abrasion that is at least >1.3X but at least one of less than <3X, <10X, <30X, <100X, <300X. As a result, the coverage density of aptamer across the sensing monolayer will vary by at least one of <30%, <100%, <300%, <1000%. Pore sizes also matter, because if the pore size is too small, aptamers bound on opposite sides of a pore can interfere with each other’s free motion in solution and/or the pores can be too small to allow proper diffusive transport of analyte such as large proteins to the aptamers. Therefore, average pore size may be at least one of >5 nm, >15 nm, >45 nm, >100 nm.

With further reference to the present invention, if too great of a porosity could disable proper functionalization of the electrode, then instead of or in addition to limiting the electrode porosity and surface area, the aptamer could be deposited in a manner such that it is reaction rate limited or diffusion rate limited inside the pore, not diffusion rate limited between the pore and aptamer-containing solution outside of feature 114. Aptamer deposition (incubation) typically requires 1-2 hours with 100′s nM aptamer concentrations in solution, a diffusion rate limited process (not reaction rate limited by thiol bonding to the gold). A simple illustrative calculation is as follows. Assume a final aptamer density on the gold surface of 1E11 aptamers/cm². Assume pores in the gold that on average have a thickness of fluid above them that is ~50 nm thick, which per cm2 would be 5E-6 cm * 1 cm² = 5E-6 mL or 5 nL. 1E11 aptamers/cm² in that 5 nL represents 1E11 aptamers / 5 nL * 1 mole / 6.02E23 aptamers * 1E9 nL / L = 33 µM of aptamer. Therefore, if aptamer solution were introduced to the pores with standard concentrations of aptamer (100′s nL to µL’s) the deposition would be diffusion rate limited through the porous network of the gold. To have the deposition rate be limited locally by diffusion rate or reaction rate inside the pores, the aptamer concentration would need to be >10 µM, and preferably > 100 µM. The above math for aptamer density, pore size, and other aspects of the aptamer deposition can be arranged such that generally aptamer concentrations in solution during deposition of the aptamer may preferably be at least one of >10, >100, >1000, >10,000 µM of aptamer. With such higher concentrations, the incubation time for deposition will be much shorter, and therefore the porous gold electrode could be dry, placed in vacuum, then dipped into the aptamer solution, and vacuum released, to rapidly introduce aptamer into the pores and avoid diffusion limited aptamer deposition, then incubated, then rapidly placed into a well-stirred buffer solution to remove aptamer as quickly as possible. Wicking into the pores of the gold is also possible if air-trapping is minimized by wicking the fluid primarily horizontally along the plane of the porous gold layer.

With further reference to the present invention, a membrane 140 as taught herein may also be applied to fill the pores 116 or only to the entrance of pores 116 where pores 116 meet area of feature 114 that would interface with tissue in the body. For example, a sensor as shown in FIG. 1B could be fabricated, dried, then coated with a membrane such as agar or polyacrylamide or collagen or cellulose membrane or crosslinked albumin, primarily only coats the entrance of pores 116 due to air entrapment inside the pores 116. Alternately pores 116 could be filled with sugar solution, dried, and sugar solution partially dissolved away in buffer solution to reveal only the entrance to pores 116, then a membrane could be coated to the entrance of pores 116 (e.g., 1′s to 10′s of nanometers thicker or even thicker such as µm’s). Alternately entrance to pores or pores themselves could be filled with a hydrogel that is then polished away after coating such that hydrogel terminated at or near the entrances of pores 116. Alternately a membrane could be coated onto much or all of the entire feature 114, including or not into the pores 116 of such feature 114, and such coating remaining on and covering at least a portion of such feature 114 even during insertion into the body.

### EXAMPLE

An example is taught with respect to creating a nano-porous electrode on a gold-plated stainless steel acupuncture needle, that can then be coated with an apatamer and be inserted into the skin and used as an electrochemcial working electrode per the present invention.

Deep Eutectic Solvent Preparation: 1:3.5 mole ratio of ZnCl:Urea was prepared in a beaker; the solution was heated to 60° C. on a hotplate while being stirred via a stir-bar until completely liquid and homogenous.

Potentiostat setup: A 3-electrode system was used for deposition. The counter wire was connected to a Zinc wire, the reference wire was connected to a flat, larger piece of zinc, and the working wire was connected to a gold-coated needle. These electrodes were immersed in the deep eutectic solvent in a beaker on a hot plate.

Electrochemical Processes: For cleaning, CV’s from 0-1.6 V were done in 0.5 M H2SO4 at a scan rate of 100 mV/s. For the nanoporous gold deposition, the gold-plated acupuncture needle was placed in the heated deep eutectic solvent. Then a -0.2 V potential was applied via chronocoulometry until a charge of 0.07 C was reached. Immediately afterwards a 1 V potential was applied via chronoamperometry until a steady anodic current of 1 µA was reached. The resulting nanoporous electrode was functionalized and tested with aptamers for cortisol and vancomycin and demonstrated to work in in-vitro with buffer or serum and in real biological tissue.

Although not described in detail herein, other steps which are readily interpreted from or incorporated along with the disclosed embodiments shall be included as part of the invention. The embodiments that have been described herein provide specific examples to portray inventive elements, but will not necessarily cover all possible embodiments commonly known to those skilled in the art.

What is claimed is:

1. A continuous sensing device for measuring at least one analyte in interstitial fluid, the device comprising:
at least one feature configured to be inserted into a body, the at least one feature configured to be inserted into a skin of the body, the at least one feature at least partially coated with at least one electrode functionalized with an aptamer sensing monolayer layer, the aptamer sensing monolayer layer comprising an aptamer with attached redox couples and passivating material,
wherein the at least one feature is configured to provide at least one of a resistance to abrasion effect or a pressure effect for the aptamer sensing monolayer when the feature is placed into the body.

2. The device of claim 1, wherein the feature comprises a porous or grooved surface, the porous or grooved surface including pores or grooves, wherein only the pores or grooves of the surface that are not exposed to tissue or cellular content are functionalized with the aptamer sensing monolayer.

3. The device of claim 1, wherein the feature comprises a membrane covering the electrode.

4. The device of claim 1, wherein at least one material is added onto the feature to provide the configuration that provides at least one of a resistance to abrasion effect or a pressure effect for the aptamer sensing monolayer when the feature is placed into the body.

5. The device of claim 3, wherein the membrane and the aptamer sensing monolayer are physically separated.

6. The device of claim 5, wherein the membrane and sensing monolayer are physically separated by at least one spacer material.

7. The device of claim 1, further comprising at least one biocompatible dissolvable material applied to the aptamer sensing monolayer.

8. The device of claim 4, wherein a biocompatible dissolvable material is the at least one material added onto the feature to provide the configuration that provides at least one of a resistance to abrasion effect or a pressure effect for the aptamer sensing monolayer when the feature is placed into the body.

9. The device of claim 1, wherein the sensing monolayer is not exposed to tissue or cellular content in the body, and the electrode coats both exposed and non-exposed portions of the least one feature inserted into the skin; and wherein the exposed portions of the electrode are coated with at least one material different from the aptamer sensing monolayer.

10. The device of claim 9, wherein the exposed portions of the at least one feature inserted into the skin are configured to absorb at least one endogenous solute found in the body.

11. The device of claim 9, wherein the at least one material different from the sensing monolayer comprises an exogeneous passivating material.

12. The device of claim 1, wherein the feature comprises an exposed area configured to be exposed to tissue or cellular content in the body after the feature is inserted into the body, and an unexposed area that is configured to be unexposed to tissue or cellular content in the body after the feature is inserted into the body, and which carriers the aptamer sensing monolayer, wherein a ratio of the unexposed to the exposed area is configured to be at least one of >1.3X,>3X,>10X,>30X,>100X.

13. The device of claim 1, wherein the feature comprises an exposed area configured to be exposed to tissue or cellular content in the body after the feature is inserted into the body, and an unexposed area that is configured to be unexposed to tissue or cellular content in the body after the feature is inserted into the body, and which carriers the aptamer sensing monolayer,
and wherein the ratio of the unexposed to the exposed area is at least one of less than <3X,<10X,<30X,<100X,<300X.

14. The device of claim 13, wherein a coverage density of aptamer across the aptamer sensing monolayer will vary by at least one of <30%, <100%, <300%, <1000%.

15. The device of claim 1, wherein the feature comprises an exposed area configured to be exposed to tissue or cellular content in the body after the feature is inserted into the body, and an unexposed area that is configured to be unexposed to tissue or cellular content in the body after the feature is inserted into the body, and which carriers the aptamer sensing monolayer,
and wherein an average pore size included in the feature is at least one of >5 nm, >15 nm, >45 nm, >100 nm.

16. A method of fabricating a continuous sensing device for measuring at least one analyte in interstitial fluid, the device comprising at least one feature inserted into a body, the feature carrying a sensing monolayer, the method comprising:
fabricating the feature configured to provide at least one of resistance to abrasion effects or pressure effects for the sensing monolayer when placed into the body.

17. The method of claim 16, further comprising coating a sensing monolayer on the at least one feature configured to be inserted into the body, and then removing the sensing monolayer from a portion of a surface of the feature.

18. The method of claim 17, further comprising coating at least one passivating material on areas where the sensing monolayer was removed.

19. The method of claim 16 further comprising coating an electrode on the at least one feature configured to be inserted into the body, and then removing the electrode from a surface of the feature, and then coating a sensing monolayer onto the remaining electrode surface.

20. The method of claim 16, further comprising coating an electrode on the at least one feature configured to be inserted into the body, and then coating a porous material onto the electrode.

21. The method of claim 16, comprising coating a sensing monolayer on the at least one feature configured to be inserted into the body; and
applying a membrane over a sensing monolayer.

22. The method of claim 16, comprising coating a sensing monolayer on the at least one feature configured to be inserted into the body;
further coating at least one spacer material onto at least a portion of the sensing monolayer; and
applying a membrane onto a least a portion of the spacer material.

23. The method of claim 16, comprising coating at least one spacer material onto at least a portion of the feature;
coating a sensing monolayer onto at least a portion of the areas of the feature not coated by the spacer material; and
applying a membrane onto a least a portion of the spacer material.

24. The method of claim 16, further comprising fabricating a plurality of pores into the feature, the plurality of pores configured to provide at least one of resistance to abrasion effects or pressure effects for the sensing monolayer when placed into the body.

25. The method of claim 24, comprising coating a membrane into at least a portion of the plurality of pores.

26. The method of claim 16, comprising coating a sensing monolayer on the at least one feature configured to be inserted into the body;
adding a membrane to the feature;
and physically separating the membrane from the sensing monolayer by at least one spacer material.

27. The method of claim 26, where the spacer material is coated onto the sensing monolayer along with a dissolvable material, and applying the membrane onto the combination of spacer material and dissolvable material.

28. The method of claim 16, wherein the sensing monolayer is fabricated using a solution with a concentration of aptamer wherein the concentration of aptamer in the solution during deposition of the aptamer is at least one of >10, >100, >1000, >10,000 µM.
